(19) United States
(12) Patent Application Publication
(10) Pub. No.: US 2005/0261344 A1
(43) Pub. Date: Nov. 24, 2005

(54) Treatment of lower urinary tract symptoms associated with overactive bladder in men and women

(76) Inventors: Charles Richard Jones, Ferrette (FR); Brian Leaker, London (GB); Michael Grant Wyllie, Herne Bay (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/523,279

(22) PCT Filed: Aug. 5, 2003
(86) PCT No.: PCT/EP03/08696

§ 371 Date: Feb. 3, 2005

(30) Foreign Application Priority Data
Aug. 14, 2002 (EP) 02255676.5

(51) Int. Cl. A61K 31/452

(52) U.S. Cl. 514/320

(57) ABSTRACT

This invention relates to pharmaceutical combinations suitable for treating the lower urinary tract symptoms associated with either overactive bladder or mild to moderate benign prostatic hyperplasia, which combinations contain an alpha-adrenoceptor antagonist and a 5-HT₄ antagonist. The combinations of the invention are particularly suitable for treating moderate or severe symptomatology.

This invention relates to pharmaceutical combinations suitable for treating the lower urinary tract symptoms associated with either overactive bladder or mild to moderate benign prostatic hyperplasia, which combinations contain an alpha-adrenoceptor antagonist and a 5-HT₄ antagonist. The combinations of the invention are particularly suitable for treating moderate or severe symptomatology.

Lower urinary tract symptoms (LUTS) are categorized by many experts as either voiding or storage symptoms. Voiding symptoms, also referred to as obstructive symptoms, include weak or intermittent urinary stream, straining when urinating, a hesitation before urine flow starts, a sense that the bladder has not emptied completely, dribbling at the end of urination or leakage afterward, painful urination, and hematuria (blood in the urine). Storage symptoms, also referred to as irritative symptoms include an increased frequency of urination, particularly at night, an urgent need to urinate, and bladder pain or irritation when urinating.

In people with an overactive bladder, the detrusor muscle (layered smooth muscle that surrounds the bladder) contracts spastically, sometimes without a known cause, which results in sustained, high bladder pressure and the urgent need to urinate (urgency). Overactive bladder results in lower urinary tract symptoms (LUTS) that affect up to one third of the adult male population. Both men and women are affected and sometimes in the case of the former, the overactive bladder is often associated with benign prostatic hyperplasia (BPH).

BPH is a progressive, nearly universal condition in aging men characterized by a nodular enlargement of prostatic tissue resulting, through obstruction of the urethra, in variable degrees of bladder outlet obstruction. As BPH progresses overgrowth occurs in the central area of the prostate called the transition zone, which wraps around the urethra and this pressure on the urethra can cause a variety of lower urinary tract symptoms (LUTS).

LUTS can also occur in the absence of prostatic obstruction. In both men and women there is a characteristic urodynamic pattern or bladder overactivity associated with the symptomatology experienced by the patient. The incidence of overactive bladder is similar in men and women, occurring in up to one third of adults.

Alpha-adrenergic receptors (herein also referred to as “alpha-adrenoceptors” or as “alpha-receptors”) are specific protein recognition sites loaded in the peripheral and central nervous systems and other tissues throughout the body. Neurotransmitters, such as noradrenaline, control many physiological processes via an action on these receptors and thereby transmit information between cells or influence cells or influence biochemical processes within the cell. Many agents capable of modifying noradrenaline activity on alpha-adrenoceptors have been developed over the last 40 years. Drugs active at alpha-adrenoceptors can be broken down into tow major classes, agonists and antagonists. Agonists, of which phenylephrine and methoxamine are examples, activate the receptor system in the same way as the endogenous neurotransmitters, adrenaline and noradrenaline. Antagonists, of which phenoxybenzamine and prazosin are examples, do not activate the receptor, but block the actions of the endogenous neurotransmitters.

Different alpha-adrenoceptor types have been discovered over the years including alpha₁-adrenoceptors and alpha₂-adrenoceptors. These receptor types are now considered to be subdivided further into subtypes including alpha_{1A, 1B, 1D, 1H, 1L} and _{1N}.

Alpha₁-adrenoceptors are known to mediate the contraction of vascular (arterial and venous) and prostatic smooth muscle. Alpha₁-adrenoceptor antagonists have been widely used as first line therapy for the treatment of hypertension and, more recently, for the symptomatic relief of BPH.

Alpha-adrenoceptor antagonists are known to relieve the obstruction by causing relaxation of the prostate smooth muscle, a decrease in urethral resistance and increased uroflow. As a result of these changes, male patients with the clinical symptoms of mild-moderate BPH experience a moderate improvement in symptoms. The magnitude of the effect is considerably less than that achieved after surgery.

Serotonin (5-HT) is another neurotransmitter that acts on receptors. Several subtypes of the 5-HT receptor have been identified and described as 5-HT₁, 5-HT₂, 5-HT₃ and 5-HT₄. 5-HT₄ antagonists have not been evaluated versus LUTS symptomatology associated with bladder overactivity in men or women, alone or in combination with other therapeutic agents.

### SUMMARY

The present invention provides a combination of an alpha-adrenoceptor antagonist and a 5-HT₄ antagonist for use as a medicament. In particular, it teaches the use of an alpha-adrenoceptor antagonist in combination with a 5-HT₄ antagonist in the manufacture of a medicament for treating the lower urinary tract symptoms. These lower urinary tract symptoms may be associated with overactive bladder or mild to moderate benign prostatic hyperplasia in mammals including man. In one embodiment, the medicament (or product) includes a first pharmaceutically acceptable composition containing an alpha-adrenoceptor antagonist and a second pharmaceutically acceptable composition containing a 5-HT₄ antagonist wherein the product is a combined preparation for simultaneous, or sequential use of the first composition and the second composition. In another embodiment of the present invention, a pharmaceutical composition is provided which comprises an alpha-adrenoceptor antagonist, a 5-HT₄ antagonist and a pharmaceutically acceptable carrier. The composition may be used in the treatment of lower urinary tract symptoms associated with overactive bladder in mammals.

In yet another embodiment of the present invention, a method of treating the lower urinary tract symptoms associated with overactive bladder or mild to moderate benign prostatic hyperplasia is provided which includes administering to a subject (or mammal) in need thereof an effective amount of an alpha-adrenoceptor antagonist in combination with a 5-HT₄ antagonist. The combination may be administered separately, simultaneously or sequentially.

### DETAILED DESCRIPTION

Reference to an alpha-adrenoceptor antagonist and/or to a 5-HT₄ antagonist shall at all times be understood to include all active forms of such agents, including the free form thereof (e.g. the free and/or base form) and also all pharmaceutically acceptable salts, polymorphs, hydrates, silicates, stereo-isomers, (e.g. diastereisomers and enantiomers) and so forth. Active metabolites of either the alpha-adrenoceptor antagonist or the 5-HT₄ antagonist, in any form, are also included.

The alpha-adrenoceptor antagonist can be selective for alpha₁-adrenoceptors or it can be non-selective, exhibiting antagonist activity at both the alpha₁ and alpha₂ receptors. Antagonists selective for the alpha₁-adrenoceptor are preferred. In the context of the known alpha₁-adrenoceptor subtypes, antagonists at _{1A, 1B, 1D, 1H, 1N} and _{1L} are equally preferred.

Suitable alpha₁-adrenoceptor antagonists include alfuzosin, indoramin, tamsulosin, doxazosin, terazosin, abanoquil, prazosin and pharmaceutically acceptable salts thereof. Preferred alpha₁-adrenoceptor antagonists include abanoquil, alfuzosin, tamsulosin, doxazosin, parvosin, terazosin, and prazosin and the pharmaceutically acceptable salts thereof, in particular doxazosin mesylate, terazosin hydrochloride and prazosin hydrocholoride.

Further alpha-adrenoceptor antagonists which are reported to be selective for the alpha₁-receptor include Recordati 15/2739, SNAP 1069, SNAP5089, RS 17053 and SL 89.0591 (Kenny et al. Expert Opin in Invest Drugs, 4, 915-923 (1995)) and/or RWJ-38,063 and RWJ-69,736 (Pulito et al. in Journal of pharmacology and experimental therapeutics, 294(1): 224-229 (2000)).

Suitable non-selective alpha-adrenoceptor antagonists include phentolamine, trazodone, dapiprazole and phenoxybenzamine. The alpha-adrenoceptor antagonists useful in this invention may be widely chosen from among those already known to the art or subsequently discovered and/or hereafter discovered and/or hereafter developed.

The alpha-adrenoceptor antagonism of a compound, and therefore its suitability for use in the present invention, can be determined using a number of conventional assays in vitro. Suitable assays include those disclosed in U.S. Pat. No. 5,599,810 which uses rabbit aorta to determine alpha₁-adrenoceptor antagonist activity and U.S. Pat. No. 5,340,814 which employ rat brain cortex to determine antagonist activity.

The 5-HT₄ antagonist can be selective for 5-HT₄ receptors or it can be non-selective, exhibiting antagonism at 5-HT₁, 5-HT₂ and 5-HT₃ receptors. Antagonists selective for the 5-HT₄ receptor are preferred.

The following compounds are known 5HT₄ receptor antagonists (This list is not exhaustive):
SB 204070: (1-butyl-4-piperidinyl)methyl 8-amino-7-chloro-1,4-benzodioxan-5-carboxylate, (Drugs of the Future, 19, 1109-1121(1994)),
SB 207266: N-[(1-butyl-4-piperidinyl)methyl[-3,4-dihydro-2H-[1,3]oxazino[3,2-a]-indole-10-carboxamide (Drugs of the Future, 22(12), 1325-1332 (1997)),
SB 207710: [(1-butyl-4-piperidinyl)methyl 8-amino-7-iodo-1,4-benzodioxan-5-carboxylate, Naunyn-Schiedeberg's Arch. Pharmacol., 349(5), 546-548 (1994)),
SB 205800: N-(1-butyl-4-piperidyl)methyl-8-amino-7-chloro-1,4-benzodioxan-5-carboxamide (WO-93/05038 as shown in Example 14),
SB 203186: 2-(1-piperidinyl)ethyl 1H-Indole-3-carboxylate (Br. J. Pharmacol., (110), 1023-1030 (1993)),
R50595: trans-4-amino-N-[1-[4,4-bis(4-fluorophenyl)butyl]-3-methoxy-4-piperidinyl]-5-chloro-2-methoxy-benzamide (Eur. J. Pharmacol., 212, 51-59 (1992))
GR 113808: 1-methyl-1H-indole-3-carboxylic acid (1-(2-((methylsulfonyl)amino)-ethyl)4-piperidinyl)methyl ester (Br. J. Pharmacol., 111, 332 (1994)), p1 GR 125487: [1-[2-[(methylsulphonyl)amino]ethyl]-4-piperidinyl]methyl 5-fluoro-2-methoxy-1H-indole-3-carboxylate (Br. J. Pharmacol., 111, 332 (1994)),
GR 138897: [1-[2-[(methylsulphonyl)amino]ethyl]-4-piperidinyl]methyl[2-(3-methyl-1,2,4-oxadiazol-5-yl)phenyl]carbamate (WO-93/20071 as shown in Examples 1 and 3),
LY-353433: 1-(1-methylethyl)-N-(2-(4-((tricyclo[3.3.1^{3,7}]dec-1-ylcarbonyl)amino)-1-piperidinyl)ethyl)-1H-indazole-3-carboxamide (Drug Dev. Res., 43(4), 193-199 (1998)),
DAU 6285: (3-endo)-8-methyl-8-azabicyclo[3.2.1]oct-3-yl-2,3-dihydro-6-methoxy-2-oxo-H-benzimidazole-1-carboxylate (Life sciences, 51(8), 583-592 (1992)),
SDZ 205-557: 2-(diethylamino)ethyl 4-amino-5-chloro-2-methoxy-benzoate (European Journal of pharmacology, 200(2-3), 373-374 (1991)),
RS 23597-190: 3-(1-piperidinyl)propyl 4-amino-5-chloro-2-methoxy-benzoate (Br. J. Pharmacol., 110(1), 119-126 (1993)).

Of the foregoing 5HT₄-antagonists the pharmaceutically acceptable salts, solvates, hydrates complexes and/or prodrugs thereof are also included in the definition of “5-HT₄ receptor antagonist”.

The 5-HT₄ antagonists useful in this invention may be widely chosen among those already known to the art or subsequently discovered and/or hereafter discovered and/or hereafter developed. In addition to those specifically identified above, 5-HT₄ antagonists have been disclosed in the patent literature, including WO-93/05038, WO-93/18036, WO-93/16072, WO-94/10174, WO-94/27965, WO-94/27987, WO-95/04737 and WO-00/37461. A particular 5-HT₄ antagonist is (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid disclosed as compound 156 in WO-00/37461, and the pharmaceutically acceptable salts thereof such as e.g. a hydrate hydrochloric acid addition salt.

The 5-HT₄ antagonist activity of a compound, and therefore its suitability for use in the present invention, can be determined using a number of conventional assays in vitro (see, Eglen et al, J Auton Pharmacol 12(5):321-333, (1992)).

A suitable combination is a 5-HT₄ antagonist and a non-selective alpha-adrenoceptor antagonist.

Preferred combinations are a 5-HT₄ antagonist with a selective alpha₁-adrenoceptor antagonist and a non-selective alpha-antagonist with a 5-HT₄ antagonist that is selective for the 5-HT₄ receptor.

A more preferred combination is a selective alpha₁-adrenoceptor antagonist and a 5-HT₄ antagonist that is selective for the 5-HT₄ receptor subtype. The most preferred is the combination of any alpha-adrenoceptor antagonist with the 5-HT₄ antagonist (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid and pharmaceutically acceptable salts thereof.

Administering both therapeutic agents produces an effect that is greater than that of either antagonist administered alone. This is advantageous in that is allows for a smaller amount of the alpha-adrenoceptor antagonist to be administered to provide a therapeutic effect. A further advantage is that therapy can be effected for patients who, for example, do not respond adequately to the use of the alpha-adrenoceptor antagonist at what would be considered maximal strength dose.

According to one aspect of the present invention, there is provided a product (medicament) comprising a first pharmaceutically acceptable composition containing an alpha-adrenoceptor antagonist and a second pharmaceutically acceptable composition containing a 5-HT₄ antagonist for use as a combined preparation for simultaneous, separate or sequential use in treating the lower urinary tract symptoms associated with overactive bladder in mammals including man.

In one embodiment, the alpha-adrenoceptor antagonist in the first composition is non-selective. Preferably the alpha-adrenoceptor antagonist in the first composition is selective for α₁-receptors. More preferably the alpha₁-adrenoceptor antagonist in the first composition is selected from doxazosin, tetrazosin, abanoquil, prazosin and indoramin and pharmaceutically acceptable salts thereof. The 5-HT₄ antagonist in the second composition may be non-selective. Preferably the 5-HT₄ antagonist in the second composition is selected from SB 205800, SB 203186, R50595, GR 113808, GR 125487, GR 138897, LY-353433, DAU 6285, SDZ 205-557, RS 23597-190, or (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid acceptable salts thereof. More preferably the 5-HT₄ antagonist in the second composition is selective for 5-HT₄ receptors. Most preferably the 5-HT₄ antagonist in the second composition is (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid and pharmaceutically acceptable salts thereof.

The present invention provides for the administering of each of the antagonists separately but as part of the same therapeutic treatment program or regimen, and it is contemplated that separate administration of each compound, at different times and by different routes, will sometimes be recommended. Thus the two components need not necessarily be administered at essentially the same time. In the preferred embodiment the alpha-adrenoceptor antagonist will be given several days prior to initiation of the 5-HT₄ antagonist either daily or “on demand”. In another preferred embodiment, administration is timed so that the peak pharmacokinetic effect of the alpha₁-adrenoceptor antagonist precedes the peak pharmacokinetic effect of the 5-HT₄ antagonist. If co-administered separately, it is also preferred that both components be administered in an oral dosage form.

The product may comprise a kit. The kit may comprise a container for containing the separate compositions such as a divided bottle or a divided foil packet, wherein each compartment contains a plurality of dosage forms (e.g. tablets) comprising either the alpha₁-adrenoceptor antagonist or the 5-HT₄ antagonist. Alternatively, rather than separating the active ingredient-containing dosage forms, the kit may contain separate compartments each of which contains whole dosage which comprises separate compositions. An example of this type of kit is a blister pack wherein each individual blister contains two tablets, one tablet comprising the alpha-adrenoceptor antagonist, the other comprising the 5-HT₄ antagonist.

Typically the kit comprises directions for the administration of the separate components. Such instructions would cover situations such as:
i. the dosage form in which the components are administered (e.g. oral and parenteral),
ii. when the component parts of the product are administered at different dosage intervals, or
iii. when titration of the individual components of the combination is desired by the prescribing physician. The container may have deposited thereon a label that describes the contents therein and any appropriate warnings.

An example of such a kit is a so-called blister pack. Blister packs are well known in the packaging industry and are widely used for the packaging of pharmaceutical unit dosage forms such as tablets, capsules, and the like. Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses between the plastic foil and the sheet. Preferably, the strength of the sheet is such that the tablets or capsules can be removed from the blister pack by manually applying pressure on the recesses thereby an opening is formed in the sheet at the place of the recess. Tablet(s) or capsule(s) can then be removed by means of the opening. It may be desirable to provide a memory aid on the kit, e.g. in the form of numbers next to the tablets or capsules whereby the numbers correspond with the days of the regimen during which the tablets or capsules so specified should be ingested. Another example of such a memory aid is a calendar printed on the card e.g. as follows “First Week, Monday, Tuesday . . . etc. . . . Second Week, Monday, Tuesday, . . . ” etc. Other variations of memory aids will be readily apparent. A “daily dose” can be a single tablet or capsule or several pills or capsules to be taken on a given day. Also a daily dose of the first compound can consist of one tablet or capsule while a daily dose of the second compound can consist of several tablets or capsules and vice versa. The memory aid should reflect this.

It is also within the scope of the present invention that both the alpha-adrenoceptor antagonist and the 5-HT₄ antagonist may be present in a single composition. Thus according to a further aspect of the invention, there is provided a pharmaceutical composition containing an a alpha-adrenoceptor antagonist, a 5-HT₄ antagonist and a pharmaceutically acceptable carrier. Suitable alpha-adrenoceptor antagonists include those that are non-selective. Preferably the alpha-adrenoceptor antagonist is selective for the alpha₁-receptor. More preferably the alpha-adrenoceptor antagonist is selected from, doxazosin, tamsulosin, alfuzosin, terazosin, abanoquil, prazosin and indoramin and pharmaceutically acceptable salts thereof. Suitable 5-HT₄ antagonists include those that are non-selective. Preferably the 5-HT₄ antagonist is selected from SB 205800, SB 203186, R50595, GR 113808, GR 125487, GR 138897, LY-353433, DAU 6285, SDZ 205-557, RS 23597-190, or (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid and pharmaceutically acceptable salts thereof. More preferably the 5-HT₄ antagonist is selective for 5-HT₄ receptors. Most preferably the 5-HT₄ antagonist in the second composition is (3S-trans)-4-(4-[[(8-chloro-3,4-dihydro-2H-benzo[b][1,4]-dioxepine-6-carbonyl)-amino]-methyl]-3-hydroxy-piperidin-1-yl)-butyric acid and pharmaceutically acceptable salts thereof.

Most preferred is a composition containing a combination of any alpha-adrenoceptor antagonist with the 5-HT₄ receptor antagonist (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid and pharmaceutically acceptable salts thereof. Preferred specific combinations include any of the alpha-adrenoceptor antagonist doxazosin, terazosin and tamsulosin with the 5-HT₄ receptor antagonist (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid and pharmaceutically acceptable salts thereof.

The compositions of the presents invention, both those that contain only one component and those that contain both, may be suitable for topical, oral, parenteral or rectal administration. The compositions may be formulated to provide immediate or sustained release of the therapeutic agent.

The compounds of the invention can be administered alone but will generally be administered as an admixture with suitable pharmaceutical excipient, diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed-, modified-, sustained-, pulsed- or controlled-release applications.

Generally tablets contain various excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatine and acacia. Additionally, lubricating agents such as magnesium stearate, stearic acid, glycerol behenate and talc may be included. Tablets may be manufactured by any standard tablet making process, for example, direct compression or a wet or dry granulation process. The tablet cores may also be coated with one or more appropriate overcoats.

Solid compositions or a similar type are also employed as fillers in gelatine capsules. Preferred excipients in this regard include lactose, milk sugar, cellulose, starch or high molecular weight polyethylene glycols. For aqueous suspensions and/or elixirs, the compounds of this invention can be combined with various sweetening agents, flavouring agents, colouring agents or dyes, emulsifying agents and/or suspending agents, diluents (e.g. water, ethanol, propylene glycol, glycerine and mixtures thereof) and combinations thereof. The compounds of the invention can also be administered parenterally, for example intravenously, intra-arterially, intraperitoneally, intracathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. If necessary, the aqueous solutions should be suitably buffered (preferably to a pH from 3 to 9). The preparation of suitable parenteral formulations under sterile conditions is readily accomplished by standard pharmaceutical techniques well-known to those skilled in the art.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene or polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-oxtyldodecanol, benzyl alcohol and water.

The alpha-adrenoceptor antagonist and/or the 5-HT₄ antagonist may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used and an auxiliary additive e.g., as a carrier, diluent or solubiliser.

Other pharmaceutical components may also be optionally included as part of the combinations useful in this invention so long as they do not interfere or adversely affect the effects of the alpha-adrenoceptor antagonist/5-HT₄ antagonist combination. The exact dose of each component administered will, of course, differ depending on the specific components prescribed, on the subject being treated, on the severity of the LUTS, on the manner of administration and on the judgement of the prescribing physician. Thus, because of patient-to-patient variability, the dosages given below are a guideline and the physician may adjust doses of the compounds to achieve the treatment the physician considers appropriate for the patient, male or female. In considering the degree of treatment desired, the physician must balance a variety of factors such as the age of the patient and the presence of other diseases or conditions (e.g. cardiovascular disease). In general, the 5-HT₄ antagonist will be administered in a range of from 0.5 to 200 mg per day, preferably 10 to 125 mg per day, more preferably 25 mg to 100 mg per day. The alpha-adrenoceptor antagonist will generally be administered in an amount of from 0.01 mg to 50 mg per day, preferably from 0.5 to 10 mg per day.

The alpha-adrenoceptor antagonist, when in combination, will be administered in the range 0.25 mg to 16 mg per day, preferably 2 mg to 4 mg per day. The 5-HT₄ antagonist will be administered twice a day in the range of 0.2 mg to 2 mg per day, preferably from 0.5 mg to 1 mg per day. All weights quoted above refer to the weight of the compounds as the free base.

Methods of preparing various pharmaceutical compositions with a certain amount of active ingredient are know, or will be apparent in light of this disclosure, to those skilled in this art. For examples of methods of preparing pharmaceutical compositions, see, Remington's Pharmaceutical Sciences, Mack Publishing Company, Easter, Pa., 15^{th} Edition (1975).

The individual components of a combination of an alpha-adrenoceptor antagonist and a 5-HT₄ antagonist can be tested in vivo in an anaesthetised beagle dog model (see Kenny et al., Urol., 44, 52-57 (1994)) in which urethral pressure and/or bladder function are measured. However, the unexpected advantage of the combination can only be determined, and thus becomes apparent on evaluation of symptoms, an assessment that can only be carried out in man.

The combination of an alpha-adrenoceptor antagonist and a 5-HT₄ antagonist can be tested clinically, typically orally, in humans. Each component is administered singly at different times to a population of male patients, each component being administered in conjunction with the International Prostate Symptom Score (IPSS) questionnaire (see, Barry et al., J Urol., 148. 1549-1563 (1992)) which evaluated patient satisfaction. Alternatively, the direct urodynamic effect of each agent, alone or in combination, on bladder overactivity, is determined in patients (McFarlane et al. Br J Urol., 80(5). 734-741 (1997).

By administering each component singly, it is meant that one component is administered, followed at a later time by the second component after having allowed an appropriate time for washout of the first component. After the washout period for each component administered singly, the components are co-administered in a manner such that both components co-operate pharmacokinetically, preferably such that fully effective drug plasma levels of both agents will be obtained. Co-administration is evaluated according to IPSS questionnaires or urodynamic profile mentioned above, thereby provided a basis for comparison of the effects of co-administration with that for each single administration. The efficacy of the present invention is demonstrated by the results of the urodynamic evaluations and/or IPSS questionnaire.

1. A pharmaceutical composition comprising an alpha-adrenoceptor antagonist, a 5-HT₄-receptor antagonist and a pharmaceutically acceptable carrier.

2. A pharmaceutical composition as claimed in claim 1 wherein the alpha-adrenoceptor antagonist is non-selective.

3. A pharmaceutical composition as claimed in claim 1 wherein the alpha-adrenoceptor antagonist is selective for the alpha₁-receptor.

4. A pharmaceutical composition as claimed in claim 1 wherein the alpha-adrenoceptor antagonist is selected from abanoquil, alfuzosin, indoramin, tamsulosin, doxazosin, parvosin, terazosin, abanoquil, prazosin and pharmaceutically acceptable salts thereof.

5. A pharmaceutical compositions as claimed in claim 1 wherein the 5-HT₄-receptor antagonist is selective for the 5-HT₄-receptor.

6. A pharmaceutical composition as claimed in claim 1 wherein the 5-HT₄-receptor antagonist is selected from SB 205800, SB 203186, R50595, GR 113808, GR 125487, GR 138897, LY-353433, DAU 6285, SDZ 205-557, RS 23597-190, or (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid and pharmaceutically acceptable salts thereof.

7. A pharmaceutical composition as claimed in claim 6 wherein the 5-HT₄-receptor antagonist is (3S-trans)-4-(4-[[(8-chloro-3,4-dihydro-2H-benzo[b][1,4]dioxepine-6-carbonyl)-amino]-methyl]-3-hydroxy-piperidin-1-yl)-butyric acid and pharmaceutically acceptable salts thereof.

8. (canceled)

9. (canceled)

10. (canceled)

11. (canceled)

12. A product comprising a first pharmaceutically acceptable composition containing an alpha-adrenoceptor antagonist and a second pharmaceutically acceptable composition a 5-HT₄-receptor antagonist for use as a combined preparation for simultaneous or sequential use in treating the lower urinary tract symptoms associated with overactive

13. A method for treating lower urinary tract symptoms comprising administering to a host in need thereof an effective amount of an alpha-adrenoceptor antagonist in combination with a 5-HT₄-receptor antagonist.

14. A method for treating lower urinary tract symptoms associated with overactive bladder comprising administering to a host in need thereof an effective amount of an alpha-adrenoceptor antagonist in combination with a 5-HT₄-receptor antagonist.

15. A method for treating the lower urinary tract symptoms associated with mild to moderate benign prostrate hyperplasia in mammals comprising administering to a mammal in need thereof an effective amount of an alpha-adrenoceptor antagonist in combination with a 5-HT₄-receptor antagonist
